(19) United States
(12) Patent Application Publication
(10) Pub. No.: US 2014/0315232 A1
(43) Pub. Date: Oct. 23, 2014

(54) METHOD FOR DOSING THE CONTROL CAPACITY OF C1INH

(71) Applicants: UNIVERSITE JOSEPH FOURIER (GRENOBLE 1), Grenoble (FR); CENTRE HOSPITALIER UNIVERSITAIRE DE GRENOBLE, Grenoble (FR)

(72) Inventors: Christian Drouet, Meylan (FR); Arije Ghannam, Grenoble (FR); Federica Defendi, La Tronche (FR); Bertrand Favier, St Vincent de Mercuze (FR)

(73) Assignee: Universite Joseph Fourier (Grenoble 1), Grenoble (FR)

(21) Appl. No.: 14/358,887

(22) PCT Filed: Nov. 28, 2012
(86) PCT No.: PCT/FR2012/052743

§ 371 Date: May 16, 2014

(30) Foreign Application Priority Data
Nov. 28, 2011 (FR) 11/60851

(51) Int. Cl. C12Q 1/37

(52) U.S. Cl. CPC C12Q 1/37
USPC 435/23

(57) ABSTRACT

The present invention relates to a method for dosing the control capacity of plasma serpin (SERine Protease INhibitor) C1 inhibitor (C1Inh) on the basis of a patient blood sample. The invention also relates to a kit specially designed for said dosing.

The present invention relates to a method for dosing the control capacity of serpin (SERineProteaseiNHibitor) C1 plasma inhibitor (C1Inh) based on a blood sample from a patient. The invention also relates to a kit specially designed for this dosing.

The scission of the high molecular weight kininogen (HK) by plasma kallikrein (KK) (also called kininoformation) leads to the release of bradykinin (BK). Bradykinin then exerts its effects by stimulating the kinin B2 receptor expressed on the endothelial cells. Serpin (SERineProteaselNhibitor) C1 inhibitor (C1Inh) is a serine protease plasma inhibitor which is involved in the control of the formation of BK. A defect in the control exerted by the C1Inh protein leads to an overproduction of BK and the angioedema (AO), being expressed by a capillary leak with swelling of the subcutaneous or submucosal tissues. Vascular permeability in the patient in crisis causes oedemas in the face and upper limbs, abdominal cramps sometimes with vomiting and diarrhea. The crisis also causes breathing difficulties which can even lead to asphyxia in the event of localization in the larynx. In some severe cases of laryngeal edema, the angioedema may cause the death of the patient.

The dosing of the control capacity of C1Inh allows diagnosing the angioedema linked to the deficit of the inhibition by C1Inh. This dosing then allows determining the quantity of C1Inh to be administered to the patient in order to re-establish the control of the formation of BK by C1Inh. It also allows a follow-up over time of the patient response to the treatment administered.

Today, the control capacity of the C1Inh protein is measured in the plasma of patients by its ability to inhibit the esterase activity of the protein C1s. This dosing is described in the article by Drouet et al, 1988. The activity C1s is determined by measuring the ethanol generated by the cleavage of a synthetic substrate of C1s, benzoyl-L-Arginine ethyl ester (BAEe). However, C1s is not a protease involved in the production of kinins, it has no impact in the pathological angioedema process. Furthermore, the main drawback in experimental terms of this dosing method is that it requires taking aliquots during incubation, thus preventing using the high throughput analysis or automated reading method.

The main control of the activation and activity of the KK is ensured by two inhibiting plasma proteins, namely, proteins C1Inh and α2-Macroglobuline (α2-M). Thus, α2-M exhibits the same control capacity on the plasma KK as the C1Inh and is hence liable to interfere in a method for the specific dosing of the inhibitor function of the C1Inh.

The present invention proposes a method for dosing the control capacity of the C1Inh protein in the plasma, which meets the aforementioned expectations.

The present invention proposes a method for dosing the control capacity of C1Inh which is correlated with the angioedema pathology and which is more specific than that described in the article by Drouet et al, 1988. More particularly, the method proposed is based on a measurement of the capacity of the KK to hydrolyze a chromogenic or fluorogenic substrate. The KK being a protease which is directly involved in the release of bradykinin, the dosing method being the object of the present invention is directly correlated to the angioedema pathology.

Furthermore, the method being the object of the present invention is performed in conditions which allow the dosing of the active C1Inh protein, while avoiding the potential activity of the α2M also present in the plasma.

Furthermore, the method of the present invention allows dosing the control capacity of the C1Inh protein, that is to say the concentration in active C1Inh, that is to say liable to exert its activity of inhibiting the release of BK. This constitutes an important advantage of the present dosing method, in particular for patients who are carriers of a mutation of the gene encoding the C1Inh protein and which makes the protein inactive. In fact, in this case, the standard tests may lead to high concentrations of C1Inh, while the protein is not active and does not play its role in the control of inhibition.

Finally, the dosing method of the present invention is carried out once, without transferring after incubation of the sample with the target protease. It is achievable on low volumes of plasma. Hence, it is applied to the automated analysis.

Dosing Method of the Control Capacity of C1Inh

The present invention hence relates to a method for dosing the control capacity of the protein C1 plasma inhibitor (C1Inh) based on a plasma sample from a patient. The dosing method according to the invention comprises the six following steps:

a) a reaction mixture is prepared based on prekallikrein (pKK), high molecular weight kininogen (HK) and activated Hageman factor protease (FXIIa), the reaction mixture being adjusted in such a manner as to have a pH higher than 7;

b) the plasma sample of the patient is incubated with a serine protease inhibitor for a duration at least equal to 5 minutes, in such a manner as to obtain a plasma sample without spontaneous protease activity and in which said inhibitor is inactivated or becomes inactive with regard to the reaction mixture prepared at step a);

c) the plasma sample obtained at step b) is incubated with the reaction mixture prepared at step a) for a duration lower than or equal to 20 minutes;

d) a chromogenic or fluorogenic substrate of the KK liable to release a chromophore or a fluorophore after hydrolysis by KK is added to the plasma sample obtained at step c);

e) the possible release of the chromophore or fluorophore obtained at step d) is detected over time; and

f) the control capacity of C1Inh is determined based on the detection achieved at step e).

The dosing method of the present invention is based on the hydrolysis of a chromogenic or fluorogenic substrate of KK, a protein of which the presence in the plasma is directly dependent on the activity of C1Inh. More particularly, the inhibitory activity of C1Inh may be dosed in the plasma of patients by measuring the amidase activity of the KK on a substrate of choice. A defect in the control of C1Inh causes an increase in the release of KK in the plasma. The plasma contains more KK and its chromogenic or fluorogenic substrate is thus highly hydrolyzed. This hydrolysis leads to the release of a chromophore or a fluorophore, the quantity of which in the sample may be detected. The quantity of chromophore or fluorophore is directly dependent on the activity of the KK and is proportional to it. However, the quantity of chromophore or fluorophore in the sample is indirectly dependent on the inhibitory activity of C1Inh and is inversely proportional to it. If the sample contains an important active concentration of C1Inh, the latter plays its role in the control of the release inhibition of KK. In this case, the plasma contains hardly any KK, the substrate thereof is hardly hydrolyzed and the quantity of fluorophore or chromophore in the sample is low.

The method of the present invention requires the preparation of a reaction mixture and putting this mixture in contact with a plasma sample of which the control capacity is sought to be evaluated by C1Inh. According to the invention, the reaction mixture contains particularly pKK and high molecular weight kininogen (HK) which associate to form the pKK/HK complex. The presence of FXIIa leads to converting the pro-enzyme pKK into an active enzyme, the KK, with, as a consequence, the cleavage of HK and release of BK. In the plasma, the activation and the activity of KK are under the control of C1Inh. C1Inh forms a covalent complex of stoichiometry 1:1 with the KK, leading to the complete loss of proteolytic and amidolytic activity. Thus, according to the present invention, the control capacity of C1Inh is measured on the hydrolyzing activity of KK. For this, a compound which is a substrate of KK is used and the hydrolysis of which releases a detectable agent (for example chromophore or fluorophore).

Thus, by “control capacity of C1Inh”, is meant an inhibitory activity or inhibitory function of the C1Inh protein. This inhibitory activity may particularly be expressed in mg/l of plasma of the patient. The C1Inh protein is a plasma inhibitor which is involved in the control of the formation of BK. A defect in the control of C1Inh leads to an increase in the concentration of BK in the plasma and potentially the angioedema. The C1Inh protein is hence directly implicated in the angioedema process.

By “patient” is meant a human being, who may be healthy or ill. The dosing method is performed based on a sample of plasma from the patient.

According to a first step of the dosing method according to the present invention, a reaction mixture is prepared based on the three following reagents: pKK, HK and activated Hageman factor protease (FXIIa).

According to the invention, the molar ratio (mol/mol) pKK/HK ranges between 1/10 and 10/1. According to an aspect of the invention, the molar ratio pKK/HK ranges between 1/2 and 2/1. According to another aspect of the present invention, the molar ratio pKK/HK of the reaction medium ranges between 3/4 and 3/2. For example, the molar ratio pKK/HK is equal to 1.

According to an aspect of the present invention, the molar ratio (mol/mol) pKK/FXIIa ranges between 1/5 and 1/25. Preferably, the molar ratio pKK/FXIIa is equal to 1/5.

The reaction medium is adjusted in such a manner as to exhibit a pH higher than 7, for example ranging between 7 and 8.5. For example, buffer solutions are used to adjust the pH of the reaction medium. By way of example, the following buffer solutions may be cited: 150 mM NaCl, 50 mM Tris-HCl, pH 7.8; 150 mM NaCl, 25 mM NaH2PO4, pH 7.6.

According to an aspect of the present invention, it is prepared a reaction mixture which exhibits a pH ranging between 7.5 and 8.0, for example a pH of 7.8

According to an aspect of the present invention, the first step of the dosing method consists in depositing a determined quantity (or a determined volume) of each reagent of the reaction mixture into a suitable recipient, for example in each well of a microtiter plate (for example, 96-well plate or 384-well plate). Thus, at the end of this step, and in as far as the recipient used is a microtiter plate, each well of the plate contains a determined identical volume of reaction mixture. Alternatively, it is prepared extemporaneously a global volume of reaction mixture, then a determined quantity (or a determined volume) of reaction mixture is deposited into a suitable recipient, for example sequentially in a 96-well plate.

According to an aspect of the invention, the reaction mixture is kept at a temperature at which proteins (that is to say, the reagents of the reaction mixture) do not or hardly exhibit any activity, for example at a temperature lower than or equal to 10° C., for example at 4° C. According to this aspect of the invention, before mixing the sample or samples of plasma from the patient with a view to dosing, the reaction mixture is incubated at a temperature which allows activating proteins during at least 5 minutes, for example during at least 10 minutes. By way of example, the reaction mixture is placed at room temperature during 10 minutes. The sub-step of this aspect of the method according to the invention allows activating the proteins of the reaction mixture. Thus, according to this aspect of the invention, the dosing method particularly comprises:
a step which consists in preparing a reaction mixture based on pKK, HK and activated Hageman factor protease (FXIIa), the reaction mixture being adjusted in such a manner as to exhibit a pH higher than 7, the mixture being kept at a temperature lower than 10° C.; and
an additional step which consists in placing the reaction mixture at room temperature during at least 5 minutes before adding the plasma sample.

According to a second step of the dosing method according to the present invention, the plasma sample from the patient is incubated with a serine protease inhibitor for a duration at least equal to 5 minutes, in such a manner as to obtain a sample of plasma without spontaneous protease activity and in which said inhibitor is inactivated or becomes inactive with regard to the reaction mixture prepared at step a). In fact, the plasma sample is liable to contain proteases, in particular KK, trypsin or elastase. Given their amidase activities, these proteases are liable to hydrolyze the chromogenic or fluorogenic substrate used within the framework of the present dosing method. In particular, the presence of KK in the plasma (or plasma KK) is liable to interfere with the dosing of the KK obtained from the pKK of the reaction mixture. Hence, it is essential to inhibit these plasma proteases, in particular, the plasma KK in as far as their activity on the substrate cannot be correlated with the presence/absence of C1Inh. In order to avoid this spontaneous activity of the plasma with regard to this chromogenic or fluorogenic substrate which is not representative of the control capacity of C1Inh, a serine protease inhibitor is used. The sample of plasma from the patient is hence incubated with a serine protease inhibitor before being incubated with the reaction medium.

The incubation of plasma with the protease inhibitor lasts at least 5 minutes. The incubation time is for example around 10 minutes, that is to say of 10+/−1 minutes. According to an aspect of the present invention, the sample of plasma is incubated with a serine protease inhibitor during a period ranging between 5 and 20 minutes. According to another aspect of the present invention, the sample of plasma is incubated with a serine protease inhibitor for at least 10 minutes.

According to an aspect of the present invention, the serine protease inhibitor is selected from among di-isopropyl fluorophosphate (DFP) or phenylmethylsulfonyl fluoride (PMSF).

According to yet another aspect of the present invention, the incubation temperature of the plasma sample with a serine protease inhibitor ranges between 25 and 40° C., for example between 25 and 35° C. The incubation temperature is for example of around 30° C., that is to say of 30+/−2° C. Such a temperature allows the inactivation of the serine protease during a short incubation period.

At step b) of the method of the present invention, the serine protease inhibitor is inactivated or becomes inactive when it enters in contact with the reaction mixture of the step a). By way of example, the DFP becomes inactive when it enters in contact with the reaction mixture exhibiting a pH higher than 7.

The operator must make sure (1) of the absence of spontaneous protease activity (complete blocking by the irreversible inhibitor) and that (2) the preparation of DFP or PMSF does not let an excess quantity of inhibitor remain, altering the measurement of the activity of the KK.

It is worth noting that according to the present invention, the order of steps a) and b) is indifferent. Step a) can be carried out first then step b). Alternatively, step b) is carried out and then step a).

According to a third step of the method of the present invention, the obtained plasma sample is incubated at the second step with the reaction mixture prepared at the first step for a duration lower than or equal to 20 minutes.

Thus, according to this step of the method of the present invention, the plasma sample, which has been incubated (or pre-incubated) in presence of the serine protease inhibitor, is incubated in presence of the reaction mixture as prepared beforehand.

Thus, according to this step of the method according to the invention, the C1Inh protein possibly present in the plasma of the patient is put in contact with the reaction mixture containing the proteins of which the activities are regulated by C1Inh.

The incubation period of the plasma sample with the reaction medium is lower than or equal to 20 minutes, for example lower than or equal to 15 minutes. The incubation period is for example of about 10 minutes, that is to say of 10+/−1 minutes. Indeed, the inventors found that by incubating the plasma with the reaction medium for a duration less than 20 minutes, it is possible to get free from the activity part of the protein α2-Macroglobuline (α2M) which ensures the control of the activation of the KK in the same way as the C1Inh. This is due to the fact that the α2M only becomes active after a certain period of incubation in the presence of the proteins that it regulates. Thus, by carrying out the dosing after an incubation period lower than 20 minutes, the interferences with the activity of the α2M are insignificant; the dosing being the object of the present invention corresponds to the only activity of C1Inh. The dosing method according to the present invention hence only corresponds to the control capacity of the C1Inh protein. This constitutes an advantageous feature of the present dosing method.

According to another aspect of the present invention, the incubation temperature of the plasma sample with the reaction medium varies between 15 and 25° C. The incubation temperature is for example that of room temperature.

According to an aspect of the present invention, a determined volume of plasma is added to each well of a microtiter plate (e.g. 96-well or 384-well) containing a defined volume of reaction medium.

According to yet another aspect of the present invention, the volume of plasma necessary for carrying out the dosing is lower than or equal to 2 μl. For example, the volume of plasma is of 0.25 μl, 0.5 μl, 0.75 μl, 1 μl, 1.25 μl, or 1.5 μl. This constitutes an advantageous feature of the present dosing method which applies to low volumes of plasma samples. The low volume of plasma required for performing the dosing, as well as the fact that this plasma sample is added to one single reaction mixture allows implementing the method in automated analysis.

According to another aspect of the present invention, the plasma volume is higher than 2 μl; for example, the plasma volume is of the order of the ml (e.g., 1 ml, 10 ml, 100 ml) or of the order of the liter. In this case, the ratio volume: molarity of the plasma: prekallikrein ranges between 1:1 and 4:1; for example, this ratio is equal to 2:1.

According to a fourth step, a chromogenic or fluorogenic substrate of the KK, liable to release a chromophore or a fluorophore after hydrolysis by KK of a chromogenic substrate of the KK, is added.

By “chromogenic substrate of the KK”, is meant a molecule able to be cleaved or modified by the KK and which comprises or is coupled to a chromophore. By “chromophore” is meant a group of atoms within a molecule which is responsible for the properties of absorption and/or emission of the light in the ultraviolet, visible or infrared domain of this molecule. These properties result from an ability to absorb the energy of photons in a range of the visible spectrum whereas the other wavelengths are transmitted or diffused. The chromogenic substrate according to the invention may be colored or colorless. This chromogenic substrate releases its chromophore under the action of the KK.

By “fluorogenic substrate” is meant a molecule able to be cleaved or modified by the KK and which comprises or is coupled to a fluorophore. This fluorogenic substrate releases its fluorophore under the action of KK. By “fluorophore”, is meant a group of atoms within a molecule which is responsible of the ability of this molecule to emit fluorescent light after excitation. They are usually substances composed of several conjugated aromatic nuclei or even planar and cyclic molecules which have one or more bonds n.

By “fluorogenic substrate”, is also meant “FRET substrate” (fluorescence resonance energy transfer), that is to say a molecule constituted of two elements (a donor fluorophore and an acceptor fluorophore) which when they are in resonance, that is to say in contact with each other before hydrolysis by the KK, emit fluorescence at a certain wavelength following an excitation. The action of the KK separates the two elements, thus leading to a loss of emission of fluorescent light at said wavelength. One of the two elements constituting the FRET substrate can, independently from the other element, emit fluorescence at a second wavelength distinct from the fluorescence emitted by the FRET substrate. Thus, the hydrolyzed FRET substrate according to the invention no longer emits fluorescence at the first wavelength but the fluorophore as an element independent from the FRET substrate emits fluorescence at a second wavelength.

The chromophores and fluorophores are known by the skilled person.

The release of the chromophore or fluorophore may be due directly or indirectly to the hydrolysis of the substrate by the KK. Thus, the KK can hydrolyze the bond coupling the substrate to the chromophore or fluorophore, thus releasing the chromophore or fluorophore from the substrate. KK may also hydrolyze a domain of the substrate not involving the chromophore or fluorophore. Preferably, the release of the chromophore or fluorophore leads to a change in color of the chromogenic substrate or a fluorescence emission from the fluorogenic substrate. As a result, the detection of the release of the chromophore or fluorophore may particularly be implemented by observing the color change of the chromogenic substrate or the fluorescence emission from the fluorogenic substrate.

Preferably, the chromogenic or fluorogenic substrate according to the invention is a derivative of a natural substrate of the KK.

According to an aspect of the present invention, the chosen substrate is a chromogenic substrate of the KK and it consists of the peptide H-D-Pro-Phe-Arg-para-nitroanilide.

According to a fifth step, the possible release of the chromophore or fluorophore obtained at the fourth step is detected over time.

This step of the dosing method of the present invention allows obtaining a kinetic of the hydrolysis activity (or amidase activity) of the KK over time. The higher the control capacity of C1Inh, the slower the hydrolysis kinetic and in particular, the lower the maximum rate of hydrolysis. On the contrary, the lower the control capacity of C1Inh, the faster the hydrolysis kinetic and in particular, the higher the maximum rate of hydrolysis.

The detection (or measurement) of the release of the chromophore or fluorophore may for example be implemented by observing the color change of the chromogenic substrate or fluorescence emission from the fluorogenic substrate.

According to an aspect of the present invention, the detection consists in a reading of the OD of the sample at the relevant wavelength.

In the case where the chromogenic substrate is the peptide H-D-Pro-Phe-Arg-para-nitroanilide, the possible release of the chromophore is detected by reading the optical deviation (OD) of the sample at a wavelength of 405 nm. In fact, the presence of the para-nitroanilide group or pNA playing the role of chromophore is detected by spectrophotometric measurement at 405 nm.

According to an aspect of the present invention, the possible release of the chromophore or fluorophore is measured based on the addition of the chromogenic or fluorogenic substrate which constitutes the time T0 and during a determined period, for example until obtaining a constant kinetics over time (kinetics value substantially constant and corresponding to a plateau phase on the kinetics curve over time). By way of example, the possible release of the chromophore or fluorophore is measured for an hour.

According to a sixth step, the control capacity of C1Inh is determined based on the detection performed at the fifth step.

This step may be performed manually or by means of a computer program, for the function Vmax (%)=∫(ng C1Inh). According to an aspect of the invention, this step consists in transferring the value of the maximum rate of hydrolysis deduced from the hydrolysis kinetics obtained at the fifth step of the dosing method on a titration curve (reference curve). By “titration curve” is meant a reference curve obtained based on maximum rate hydrolysis measurements carried out with known quantities of C1Inh protein, for example ranging between 40 ng and 200 ng of C1Inh protein.

According to another aspect of the invention, this step consists in entering the value into a computer program and deducing the control capacity of C1Inh, for example by means of an algorithm.

The control capacity of C1Inh may for example be expressed in mg/l or in U/ml (U=20 U/ml), U being the arbitrary unit such as defined in the article by Drouet et al., 1998.

Kit or Case for Implementing the Dosing

The present invention also relates to a kit or case for implementing the method according to the invention, comprising:
pKK, HK and activated Hageman factor protease (FXIIa),
solutions allowing obtaining a pH higher than 7,
serine protease inhibitor, and
a chromogenic or fluorogenic substrate of KK.

The kinetic measurement of the method may be automated within the scope of its application in high throughput analysis systems.

### Other Methods

The present invention also relates to a method for determining the quantity of plasma C1Inh required to stop the effects of an angioedema in a patient, comprising:

a) dosing the control capacity of the C1Inh protein based on a sample of plasma from the patient, according to the invention, and

b) determining the quantity of C1Inh to be administered to the patient.

The present invention also relates to a method for determining the quantity of plasma C1Inh stimulator required to stop the effects of an angioedema in a patient, comprising:

a) dosing the control capacity of the C1Inh protein based on a sample of plasma from the patient, according to the invention, and

b) determining the quantity of C1Inh stimulator to be administered to the patient.

The step b) of the two determination methods being also the object of the present invention, may be carried out by comparison with values of reference (healthy patient or same patient in non pathological situation).

The present invention also relates to a method for monitoring the control capacity of C1Inh in a patient over time consisting in dosing at two different times the control capacity of C1Inh based on a sample of plasma from this patient according to the dosing method of the present invention.

This monitoring method may be particularly set up in the following situations: a) after administering drugs intended for prophylaxis of the angioedema (biosynthesis inductor such as danazol and stanazolol); b) after substitution therapy in situations of acquired angioedema treated by purified or recombinant C1Inh; c) after administering C1Inh concentrates applied in inflammatory situations (sepsis) or to counter the adverse effects of antihypertensive therapy (inhibitors of the angiotensin converting enzyme-I; sartans); d) after administering gliptins within the framework of type II diabetes treatment, e) during alopecia treatment or in the aftermath of surgery of prostate tumors (inhibitors of the 5α-reductase).

### DESCRIPTION OF THE FIGURES

FIG. 1: Formation of endothelial bradykinin (kininoformation). C1Inh has a strategic position in the control of the kininoformation.

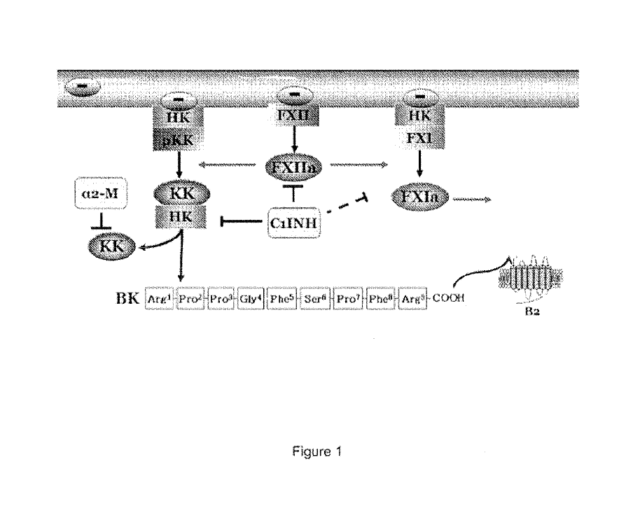

FIG. 2: Titration curve of the control function of C1Inh. The kinetics of the activation of the pKK (Vmax) is measured in the presence of increasing doses of C1Inh. Incubation of 10 minutes.

(A) Kinetics of the amidase activity as a function of time. On the x-axis, absorbance (OD at 405 nm); on the y-axis, time in minutes.

(B) Titration of the function of C1Inh by the Vmax of the activation of the pKK. On the x-axis, Vmax in nmol·ml⁻¹·min⁻¹; on the y-axis, FPH samples with/without C1Inh.

FPH: Incubation product of Factor XII (F)+Prekallikrein or PKK (P)+high molecular weight Kininogen or HK (H).

FIG. 3: Titration curve of the function of the α2M. The kinetics of the activation of the pKK (Vmax) is measured in the presence of increasing doses of α2M.

(A) Kinetics of the amidase activity as a function of time. On the x-axis, absorbance (OD at 405 nm); on the y-axis, time in minutes.

(B) Titration of the function of the α2M by the Vmax of the activation of the pKK. On the x-axis, Vmax in nmol·ml⁻¹·min⁻¹; on the y-axis, FPH samples with/without α2M.

FIG. 4: Maximum rate of the activation kinetics of the pKK in the presence of increasing volumes of plasma not subjected to the pre-incubation by the DFP (A) and subjected to the pre-incubation by the DFP (B). On the x-axis, Vmax in nmol·ml⁻¹·min⁻¹; on the y-axis, FPH samples with/without plasma with/without DFP.

FIG. 5: (A) Reproducibility of the inhibition test of the activation of pKK by increasing concentrations of C1Inh. (B) Titration curve of C1 Inh. Increasing quantities of C1Inh are applied in the activation test of the pKK, the residual Vmax Vmax % is calculated with the ratio Vmaxexp/Vmax 100 and the curve Vmax (%)=∫(ng C1Inh) is plotted for the series of measurements of the function of C1Inh in the samples.

FIG. 6: Measurement of the function of C1Inh in a patient carrying the mutation Arg444Ser. (A) Repeatability of the measurements of the Vmax in the presence of the sample (patient and control). (B) Set of calibration solutions with the values of the residual Vmax observed in A.

### EXAMPLES

### Materials and Methods

1. Sampling

2×4.5 mL of blood are drawn by venipuncture and collected in tubes containing sodium citrate (0.1 mol/L). The plasma is collected after centrifugal process (22° C., 10 min, 2500 g). The plasma is aliquoted into volumes of 0.5 ml and frozen at −80° C.

2. Protease and Proteins Used

The activated Hageman factor protease (FXIIa), the prekallikrein (pKK), high molecular weight kininogen (HK) come from the company Enzyme Research Laboratories Ltd (Swansea UK).

2 pmol of pKK correspond to 150 ng of protein (MW 75 000 Da).

2 pmol of HK correspond to 206.25 ng of protein (MW 110 000 Da).

The set of calibration solutions is established based on purified C1Inh (BERINERT®, CSL Behring).

2 pmol of C1Inh correspond to 200 ng of purified C1Inh (MW 105 000 Da).

The human α2M comes from BIOMAC (Leipzig, Germany).

0.4 pmol of α2M correspond to 300 ng of protein (MW 720 000 Da).

The activity of the KK is evaluated by an amidolytic method using a chromogenic substrate of the KK, the tripeptide H-D-Pro-Phe, Arg-pNA (residues P3-P1 of the scission of kininogen, accession ID P01042, positions 387-389). The hydrolysis of the para-nitroanilide group (pNA) by the KK is detected by spectrophotometric measurement at 30° C. at 405 nm (ε405 nm=8800 M⁻¹·cm⁻¹). This tripeptide comes from the company BACHEM.

3. Preparing the Reaction Mixture

The reaction mixture (also called FPH) is prepared as described hereinafter: each well of the microtiter plate is saturated beforehand by 1% PEG 6000 for 1 h at room temperature. In each well of a microtiter plate (96-well plate) at 4° C., 150 ng of pKK and 206.25 ng of HK (ratio mol/mol of pKK/HK=1), then 25 ng of protease FXIIa are successively deposited. It is preferable to keep the microtiter plate at 4° C. in such a manner as to block the activity of the proteins in the reaction mixture. The final volume in each well is equal to 230 μl, adjusted with buffer Tris NaCl (NaCl 150 mM, Tris-HCl 50 mM) in such a manner that the reaction mixture exhibits a pH of 7.8.

Before adding the C1Inh or the plasma sample, the plate is subjected to a 10 minutes pre-incubation at room temperature.

4. Preparing a Titration Curve of the Control Capacity of C1Inh

The concentration range of C1Inh is established in decreasing concentration starting from the stoichiometric concentration 1/1 (mol/mol) between C1Inh and pKK.

In each well of the microtiter plate (96-well plate) prepared according to paragraph 3 above, quantities of 200 ng, 160 ng, 120 ng, 80 ng and 40 ng of C1Inh (estimation in quantity of protein) are deposited. The mixture is then subjected to a pre-incubation period of 10 minutes at 30° C.

The reaction is triggered by adding the tripeptide H-D-Pro-Phe-arg-pNA (0.83 mM final), and the absorbance is followed for 60 minutes at 30° C. at 405 nm on the THERMOFISCHER MULTISKAN GO apparatus.

5. Impact of the α2-Macroglobuline on the Activation of the Contact Phase

In each well of the microtiter plate (96-well plate) prepared according to the above paragraph 3, are deposited decreasing concentrations of:
α2M alone (300 ng, 240 ng, 180 ng, 120 ng and 60 ng; namely quantities corresponding to volumes of 0.05-0.5 μl of human plasma), and
a mixture of C1Inh and α2-M (decreasing concentrations of each serpin).

The mixture is subjected to incubation for 10 minutes at 37° C.

The reaction is triggered by adding the tripeptide H-D-Pro-Phe-Arg-pNA (0.83 mM final), and the absorbance is followed for 60 minutes at 30° C. at 405 nm on the THERMOFISCHER MULTISKAN GO apparatus.

6. Volumes of Plasma Required, Presence of Serine Protease Inhibitor

First, volumes of plasma from a patient or healthy subject (control) varying between 0.25 and 1 μl are deposited in each well of the microtiter plate (96-well plate) prepared according to the above paragraph 3.

Second, the dosings are repeated after incubation with di-isopropyl fluorophosphate (DFP), a serine protease inhibitor. Indeed, in order to avoid the presence of KK in the plasma of the patient (also called spontaneous activity of the plasma) with respect to the tripeptide H-D-Pro-Phe-Arg-pNA, the plasma samples are subjected to pre-incubation in presence of 0.5 mM of DFP (Sigma), at room temperature during 10 minutes. The DFP blocks the enzymatic activities of the serine proteases, in particular KK, trypsine and elastase, which would be present in the plasma and could interfere with the dosing (spontaneous activity of the serine proteases, in particular KK). The use of DFP is possible within the scope of the present dosing method due to the fact that this inhibitor is hydrolyzed when it is in contact with a medium exhibiting a pH higher than 7 and optionally having amine functional groups.

The mixtures are subjected to the incubation for 10 min at 37° C.

The reaction is triggered by adding the tripeptide H-D-Pro-Phe-Arg-pNA (0.83 mM final), and the absorbance is followed for 60 minutes at 30° C. at 405 nm on the THERMOFISCHER MULTISKAN GO apparatus.

### Result and Discussion

C1Inh Titration Curve, Incubation Time of C1Inh in the Reaction Mixture

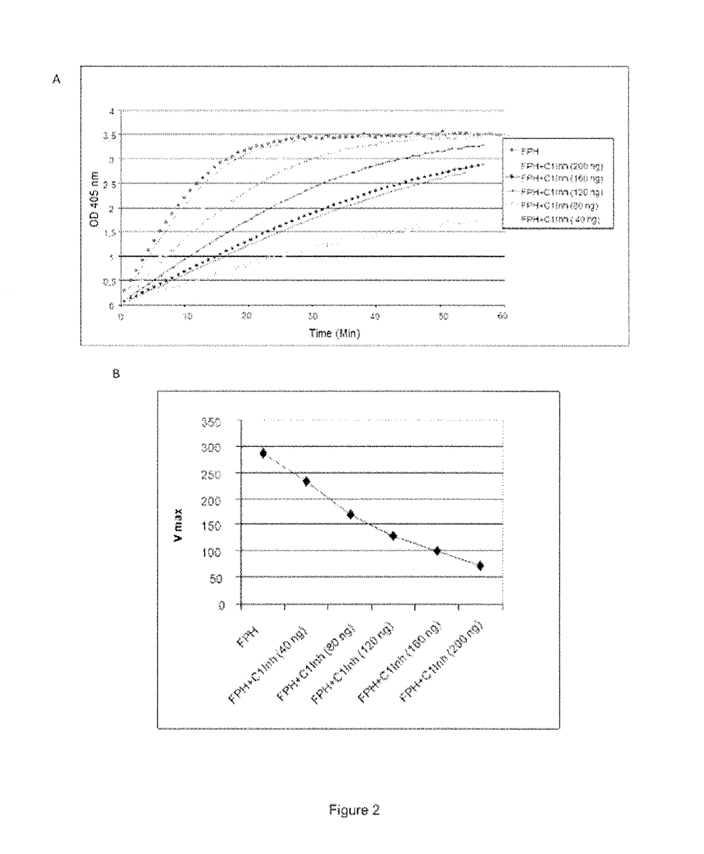

FIG. 2A shows the results of the kinetics at the different concentrations of C1Inh.

The measurement of the most favorable Vmax is obtained for the incubation period of 10 minutes at 37° C. (results not shown). The maximum rate is measured for each concentration.

As is shown in FIG. 2B, the decrease of the Vmax is quasi-linear with the increase of the concentration of C1Inh (40-200 ng): from 233 nmol·ml⁻¹·min⁻¹ (for 40 ng of C1Inh) to 68 nmol·ml⁻¹·min⁻¹ (for 200 ng of C1Inh). The control carried out without C1Inh gives a Vmax of 280 nmol·ml⁻¹·min⁻¹.

Titration Curve of α2-Macroalobuline (α2M) Alone or in Presence of C1Inh

Knowing the possible control of the activation of the pKK by the α2M, and due to the high concentration of serpin α2M in the plasma, its impact on the system has been tested with concentrations 2 times higher than that of C1Inh in order to comply with the conditions of human plasma.

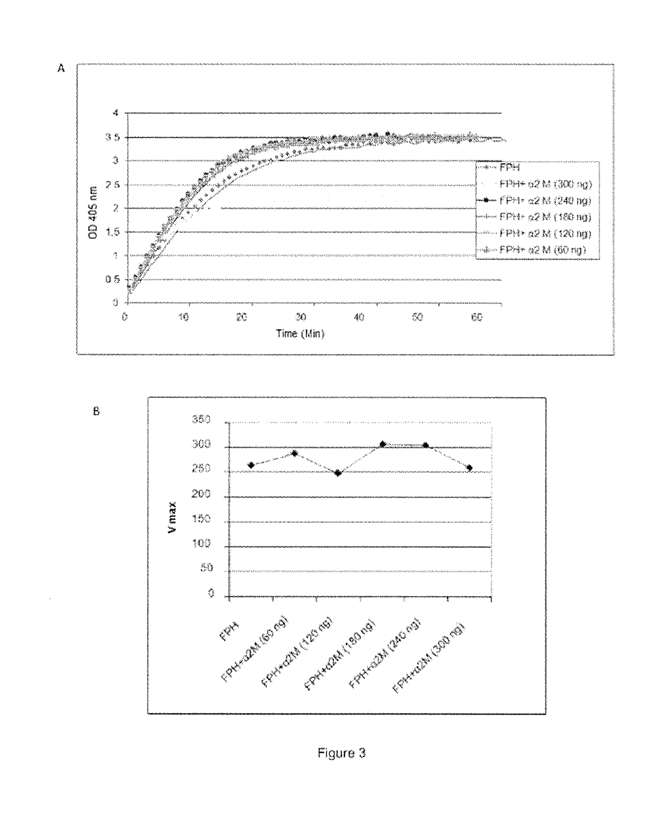

FIG. 3, in particular FIG. 3B, show that, whatever the concentration of the α2M, the Vmax is in the range of 260 nmol·ml⁻¹·min⁻¹, value observed regularly as that of the activation of the contact phase in the retained conditions. This value is hence not modified by adding increasing doses of the α2M. The impact of the α2M on the activation of the contact phase is insignificant for the incubation period of 10 minutes.

In order to show that the control function of the activation of the pKK by C1Inh, target of the test, is not affected by the presence of the other serpin α2M, the same test as before is carried out in the presence of the mixture of the two serpins C1Inh and α2M and by respecting the ratio C1Inh:α2M of 1:2 (per mol). The Vmax decreases in a quasi-linear manner as for FIG. 2, without added effect of the α2M (results not shown). Compared with the results of the activation kinetics of the pKK in the absence of the α2M, the effect of the α2M is not detectable. This confirms the data on FIG. 3.

As a consequence, in the incubation period of 10 min, the impact of the α2M on the control of the activation of the pKK is insignificant.

Evaluation of the Optimal Quantity of Plasma for Measuring the Control by C1Inh

The same test as before has been carried out in the presence of the plasma of healthy subjects and patients for whom the function of C1Inh has been known to be lowered. Increasing volumes (0.25-10 μl) have been applied instead of adding either one of the serpins.

The equivalent C1 Inh, used in the test, of the plasma of healthy subjects is of 1 μl of plasma representing 200 to 300 ng of C1Inh. This situation corresponds to the lowest point of the Vmax (FIG. 2B).

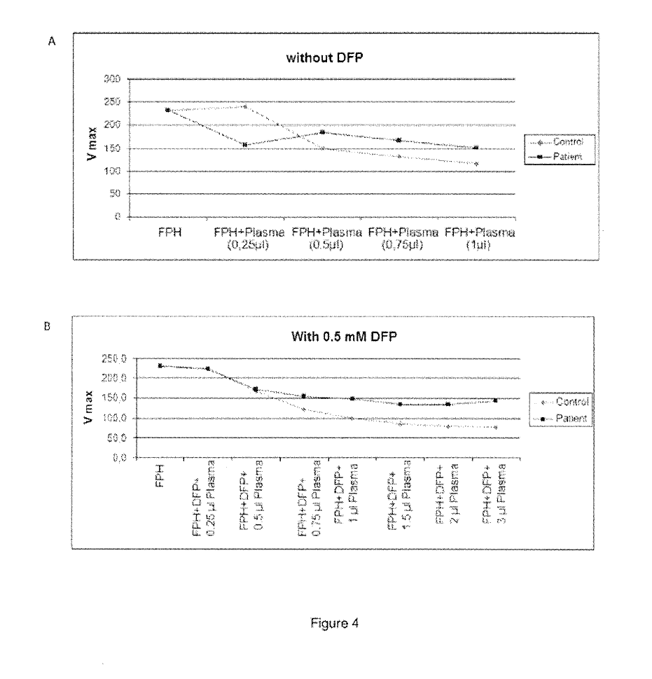

The FIG. 4A shows that the application of the volume of 0.5 μl of plasma of the patient increases the Vmax. This increase is attributed to the strong spontaneous kininogenase activity (74 nmol·ml⁻¹·min⁻¹; reference 2.4-10.7 nmol·ml⁻¹·min⁻¹). Such as shown in FIG. 4B, this activity is inhibited by the pre-incubation of the plasma with 0.5 mM of DFP during 10 min at room temperature. This pre-incubation does not modify the Vmax of the activation of the pKK in the presence of the plasma. The DFP does not disrupt the activation of the KK, nor the control function by C1Inh in the conditions of the test (FIGS. 4A and B).

Reproducibility of the Results, Preparing a Titration Curve and Applying to Patient Samples

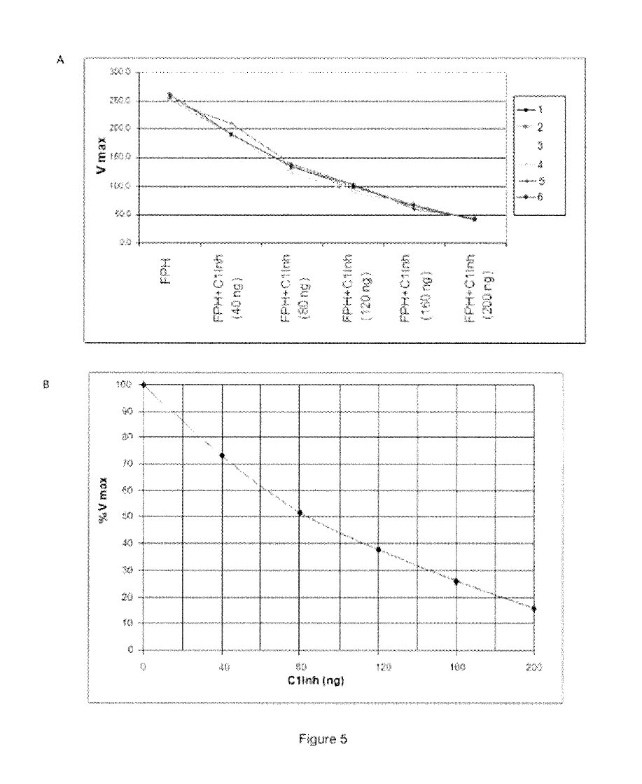

The inhibition test of the activation of pKK by increasing concentrations of C1Inh has been reproduced in 6 independent tests. See FIG. 5A. The values obtained are all grouped around the average with variability of 2.5 to 5.5%.

Data taken from the Vmax (nmol·ml⁻¹·min⁻¹)

For each quantity of added C1Inh: (0) average 256.9, standard deviation 7.6; (40 ng) average 196.7, standard deviation 9.31; (80 ng) average 132.6, standard deviation 5.45; (120 ng) average 97.4, standard deviation 5.39; (160 ng) average 65.5, standard deviation 2.97; (200 ng) average 42.2, standard deviation 1.08.

The addition of increasing concentrations of C1Inh leads to the quasi-linear decrease of the Vmax. Thus, this allows considering the addition of C1Inh in the set of calibration solutions of the activation test of the pKK in the presence of the plasma.

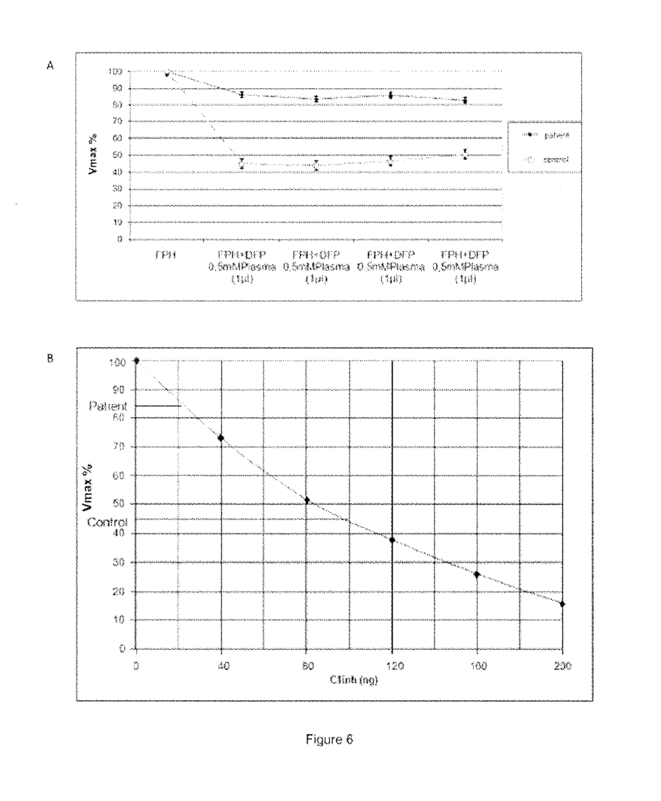

As for the titration curve Vmax (%)=∫(ng C1Inh) (FIG. 5B), it is plotted by using the averages of the 6 series of measurements obtained with known quantities of C1Inh (FIG. 6A). The residual Vmax Vmax % is calculated with the ratio Vmaxexp/Vmax 100. Thus, for example, in the sample FPH+C1Inh 80 ng, a value of Vmax of 132.6/256.9×100=51.6% is obtained.

The low volume of plasma and the addition of the components in one single reaction medium allow using the method for automated chain applications. The application of the dosing method is also possible by developing the procedure with more important volumes, provided that the constraints of concentration of proteins Factor Xlla, PKK and HK (FPH) and of the ratio 2:1, plasma:pKK (Vol:Mol) are respected.

Comparative Dosing of the Control Capacity of C1Inh by the Test of the Prior Art and by that of the Present Invention

a) Subject

The patient is a female subject aged 68, with HAE type II and carrier of the mutation Arg444Ser on C1Inh. The mutant is considered as unable to control the activity of protease KK.

b) C1Inh protein concentration

This patient exhibits an antigenemia (or concentration) of C1Inh: 503 mg/l (NB: reference values: 210-345 mg/l). The antigenemia is measured by nephelometry (nephelometer Dade Behring BN II).

From this measurement, it is deduced a high concentration in C1Inh protein. Considering that the patient is nonetheless a carrier of a mutation Arg444Ser on C1Inh, one has to know what the percentage of unmutated C1Inh is, that is to say active protein, in the blood of the patient.

c) Control capacity of C1Inh dosed by means of the technique of inhibition of the esterase activity of the protease C1s.

The inhibitory activity of C1Inh has been measured by means of the test of the prior art described in the article by Drouet et al., 1988 (technique of inhibition of the esterase activity of the protease C1s). The measurement of the function performed by the method of the present invention turns out to be possible in scales of values lower than the detection threshold, that is to say <2 U/ml, or <30 mg/L (NB: reference values 17.2-27.4 U/ml).

d) Control capacity of C1Inh dosed by means of the method of the present invention

It is deduced from FIG. 6A that the Vmax of the patient is of 85% and the Vmax of the control is of 45%. These two V-max values transposed onto the titration curve of FIG. 6B allow evaluating the equivalent activity of C1Inh,

The patient develops the equivalent activity C1Inh of 22 ng, for an experimental control of 100 ng. The function of plasma C1Inh is hence of 22%. The equivalent activity C1Inh of 22 ng is that measured in a volume of 1 μl and hence corresponds to a concentration of 22 mg/l.

Thus the measurement carried out by the method of the present invention leads to a control capacity value of C1Inh of 22 mg/l. This value is to be compared to the value obtained according to the technique of inhibition of the esterase activity of the protease C1s, <30 mg/L. The dosing technique of the prior art does not allow obtaining a precise value of the control capacity of C1Inh in such low scales. Thus, the test of the prior art does not allow monitoring the progress over time of the control capacity of C1Inh.

Furthermore, it is noted that the dosing carried out by means of the anti-C1Inh antibodies indicates a C1Inh concentration of 503 mg/l. Thus, among all the C1Inh molecules present in the plasma of the patient, only a small portion is active, around 4%.

Note: For the volume of 1 μl, it is expected that the measurement of Vmax is associated with the C1Inh equivalent antigenemia value of 200 ng. The residual experimental Vmax is equal to 100 ng namely half the expected value.

This experimental value is half of that anticipated by the titration curve; to explain this observation the formulated hypothesis is the absence of direct correlation between the purified C1Inh protein (used in the titration curve) and the plasma C1Inh protein (engaging bonds with plasma proteins, with loss of part of its reactivity).

1. A method for dosing the control capacity of the protein C1 plasma inhibitor (C1Inh) based on a sample of plasma from a patient, comprising the following steps:
a) a reaction mixture is prepared based on prekallikrein (pKK), high molecular weight kininogen (HK) and activated Hageman factor protease (FXIIa), the reaction mixture being adjusted in such a manner as to have a pH higher than 7;
b) the plasma sample of the patient is incubated with a serine protease inhibitor for a duration at least equal to 5 minutes, in such a manner as to obtain a plasma sample without spontaneous protease activity and in which said inhibitor is inactivated or becomes inactive with regard to the reaction mixture prepared at step a);
c) the plasma sample obtained at step b) is incubated with the reaction mixture prepared at step a) for a duration lower than or equal to 20 minutes;
d) a chromogenic or fluorogenic substrate of the kallikrein (KK), liable to release a chromophore or a fluorophore after hydrolysis by KK, is added to the plasma sample obtained at step c);
e) the possible release of the chromophore or fluorophore obtained at step d) is detected over time; and
f) the control capacity of C1Inh is determined based on the detection achieved at step e).

2. The dosing method according to claim 1, according to which the ratio (mol/mol) pKK/HK of the reaction mixture ranges between 1/10 and 10/1, preferably for example between 1/2 and 2/1.

3. The dosing method according to claim 1, according to which the reaction mixture exhibits a pH ranging between 7 and 8.5.

4. The dosing method according to claim 1, according to which the serine protease inhibitor is selected from among di-isopropyl fluorophosphate (DFP) or phenylmethylsulfonyl fluoride (PMSF).

5. The dosing method according to claim 1, according to which at step d) the peptide H-D-Pro-Phe-Arg-para-nitroanilide is used as chromogenic substrate of the KK and at step e) the possible presence of the chromophore pNA is detected by spectrophotometric reading at 405 nm.

6. The dosing method according to claim 1, according to which the volume of plasma required to perform the dosing is lower than 2 μl.

7. A kit for implementing the method according to claim 1, comprising:
prekallikrein (pKK), high molecular weight kininogen (HK) and activated Hageman factor protease (FXIIa),
solutions allowing obtaining a pH higher than 7,
a serine protease inhibitor, and
a chromogenic or fluorogenic substrate of KK.

8. A method for determining the quantity of plasma C1Inh required for stopping the effects of an angioedema in a patient, comprising:
a) dosing the control capacity of the C1Inh protein based on a sample of plasma from the patient, according to claim 1, and
b) determining the quantity of C1Inh to be administered to the patient.

9. A method for determining the quantity of plasma C1Inh stimulator required for stopping the effects of an angioedema in a patient, comprising:
a) dosing the control capacity of the C1Inh protein based on a sample of plasma from the patient, according to claim 1, and
b) determining the quantity of stimulator of C1Inh to be administered to the patient.

10. A method for monitoring the control capacity of C1Inh in a patient over time consisting in dosing at two different times the control capacity of C1Inh based on a sample of plasma from this patient according to the method of claim 1.
